(19) United States
(12) Patent Application Publication
(10) Pub. No.: US 2002/0129392 A1
(43) Pub. Date: Sep. 12, 2002

(54) Immunocompetent animals including xenogeneic implants of mesenchymal stem cells

(75) Inventors: Padmavathy Vanguri, Cockeysville, MD (US); Robert Deans, Claremont, CA (US)

Correspondence Address: CARELLA, BYRNE, BAIN, GILFILLAN,, CECCHI, STEWART & OLSTEIN, 6 Becker Farm Road, Roseland, NJ, 07068, US

(73) Assignee: Osiris Therapeutics, Inc.

(21) Appl. No.: 09/992,229

(22) Filed: Nov. 14, 2001

Related U.S. Application Data
(60) Provisional application No. 60/248,812, filed on Nov. 15, 2000.

(51) Int. Cl. A01K 67/027

(52) U.S. Cl. 800/14; 800/18; 800/3

(57) ABSTRACT

An immunocompetent post-natal animal of a first species having had administered thereto, such as by implantation or injection, mesenchymal stem cells which were obtained from an animal of a second species. In one embodiment, an immunocompetent mouse has human mesenchymal stem cells implanted therein.

This application is a continuation-in-part of, and claims priority based on, provisional application Serial No. 60/248,812, filed Nov. 15, 2000, the contents of which are incorporated by reference in their entirety.

This invention relates to immunocompetent post-natal animals having xenogeneic implants. More particularly, this invention relates to immunocompetent animals having xenogeneic implants of mesenchymal stem cells, or MSCs. In one embodiment, there is provided an immunocompetent mouse which includes one or more implants of human mesenchymal stem cells.

Mesenchymal stem cells (MSCs) are multipotent cells which may be derived from bone marrow and other tissues. MSCs, including human MSCs (hMSCs) differentiate into several mesenchymal lineages including adipogenic (U.S. Pat. No. 5,827,740), osteogenic (U.S. Pat. No. 5,736,396), chondrogenic (U.S. Pat. No. 5,908,784) and tenogic (U.S. Pat. No. 5,855,619), under appropriate in vitro or in vivo conditions. These cells can be expanded extensively in vitro and still maintain their multipotent stem cell phenotype.

MSCs are easily and efficiently transduced with retroviral vectors as demonstrated previously with GFP and human IL-3. (Mol. Ther., Vol. 3, pgs. 857-866(2001)). The transduced MSCs express significant amounts of the transgene product in culture in vitro and upon delivery by several different routes in vivo. Furthermore, the transduced MSCs maintain transgene expression after differentiation in vitro or in vivo. (U.S. Pat. No. 5,591,625) These characteristics make them an attractive cellular gene delivery vehicle. Previously it was demonstrated that hMSCs transduced with human erythropoietin (Epo), express high levels of human Epo in vitro. When transduced cells are injected into the muscle of immunodeficient nod/SCID mice, human Epo is measurable in the plasma and induces an increase in the hematocrit of the mice for extended periods. (Mol. Ther., Vol. 1, pg. 583 (2000)) Similarly, other genes including soluble TNFRII (Mol. Ther., Vol. 1, pg. S197 (2000)) and BMP-7 (Mol. Ther., Vol. 1, pg. S143 (2000)) have been studied using MSCs as vehicles of gene delivery. hMSCs have also been transduced with a lysosomal enzyme alpha-galactosidase A (αGAIA), towards a potential therapy for Fabry disease. Implantation of these cells in NOD/SCID mice showed an increase in αGAIA in the plasma of these mice and the cells containing the transgene product were detected in the injected muscle two weeks after injection. (Blood, Vol. 96, pg. 845a (2000)). An excellent model to study efficacy of αGAIA-MSCs for treatment of Fabry disease is the Fabry knock-out mouse model. Similarly several transgenic, mutant or knock-out mice serve as excellent animal models for various human diseases; however, mouse MSCs cannot be routinely isolated and their similarity to hMSCs is not clear. In order to avoid rejection of human cells, sever combined immune deficiency (SCID) and athymic rats have been used; however, results obtained in these experiments are impacted by the lack of immune response.

In vitro experiments using allogeneic combinations of MSCs and peripheral blood mononuclear cells (PBMCs) showed that MSCs exhibit very low immunogenicity and actively suppress T cell responses. (McIntosh, et al., Manuscript in preparation.) Same-species allogeneic MSCs have been successfully used in baboons (Blood, Vol. 97, No. 11, pg. 2718 (2001)), and pigs (Circulation, in press) for regeneration of bone or heart tissue, respectively.

Human MSCs have been shown to engraft and differentiate when administered in utero to fetal sheep. (Nature Medicine, Vol. 6, No. 11, pgs. 1282-1286 (November 2000)).

Transplantation of xenogeneic cells has been investigated by several groups in the context of gene or protein delivery. In almost all cases some kind of immuno-isolation or encapsulation of the xenogeneic cells together with immunosuppression of the recipient animals was necessary. As an exception, encapsulated xenogeneic cells survived as long as 6 months in the absence of immunosuppresion when implanted in the central nervous system (CNS) presumably due to the immunoprivileged status of the CNS. (Nature Medicine, Vol. 2, pg. 696 (1996); Anesthesiology, Vol. 85, pg. 1005 (1996)). Among several cell types studied, primary islet xenografts appear to be the least antigenic. Xenogenic islets have been reported to survive after intraperitoneal or subcutaneous implantation in the absence of immunosuppression, but only if they are encapsulated. (Proc. Nat. Acad. Sci., Vol. 88, pg. 11100 (1991); Science, Vol. 254,pg 1782 (1991). Unencapsulated islets survived only after intrahepatic pre-immunization and with transient immunosuppression. (J. Clin. Invest., Vol. 93, pg. 1313 (1994).

The invention now will be described with respect to the following drawings, wherein:

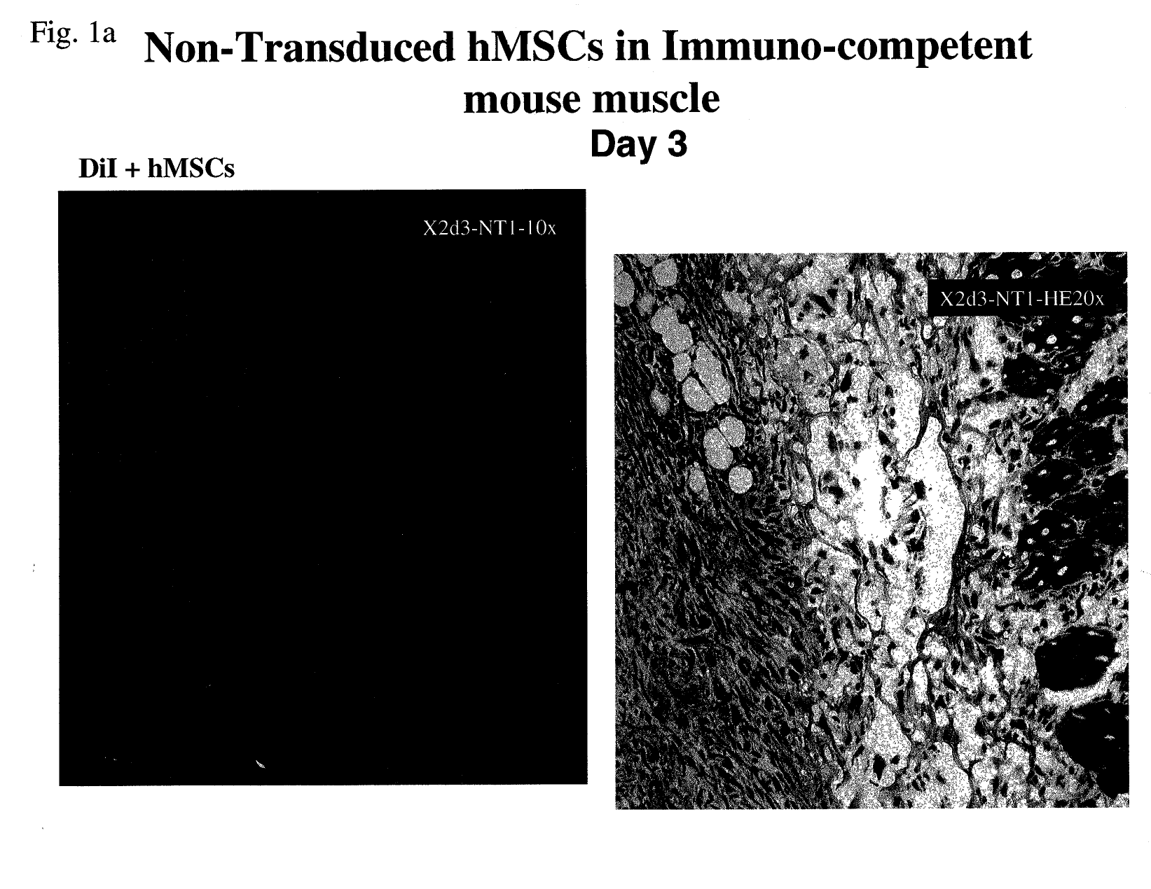

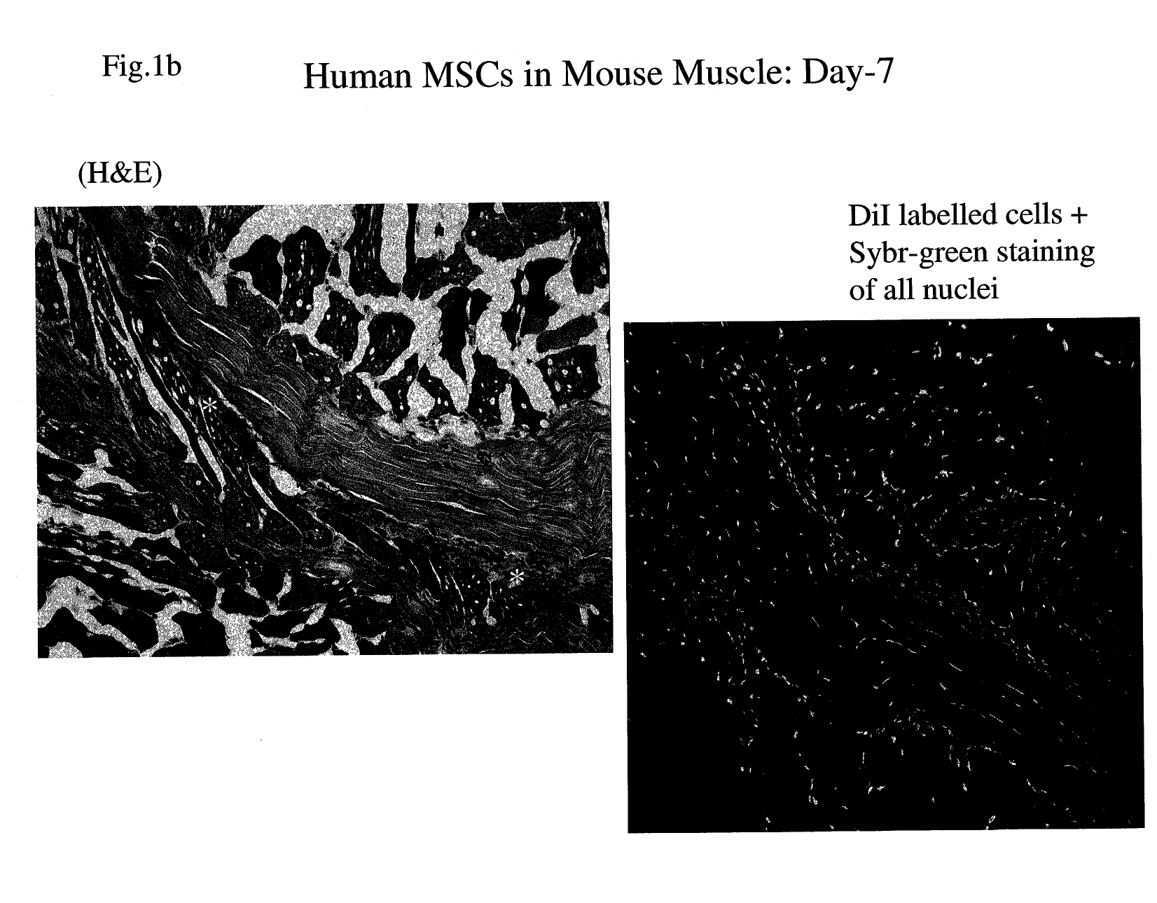

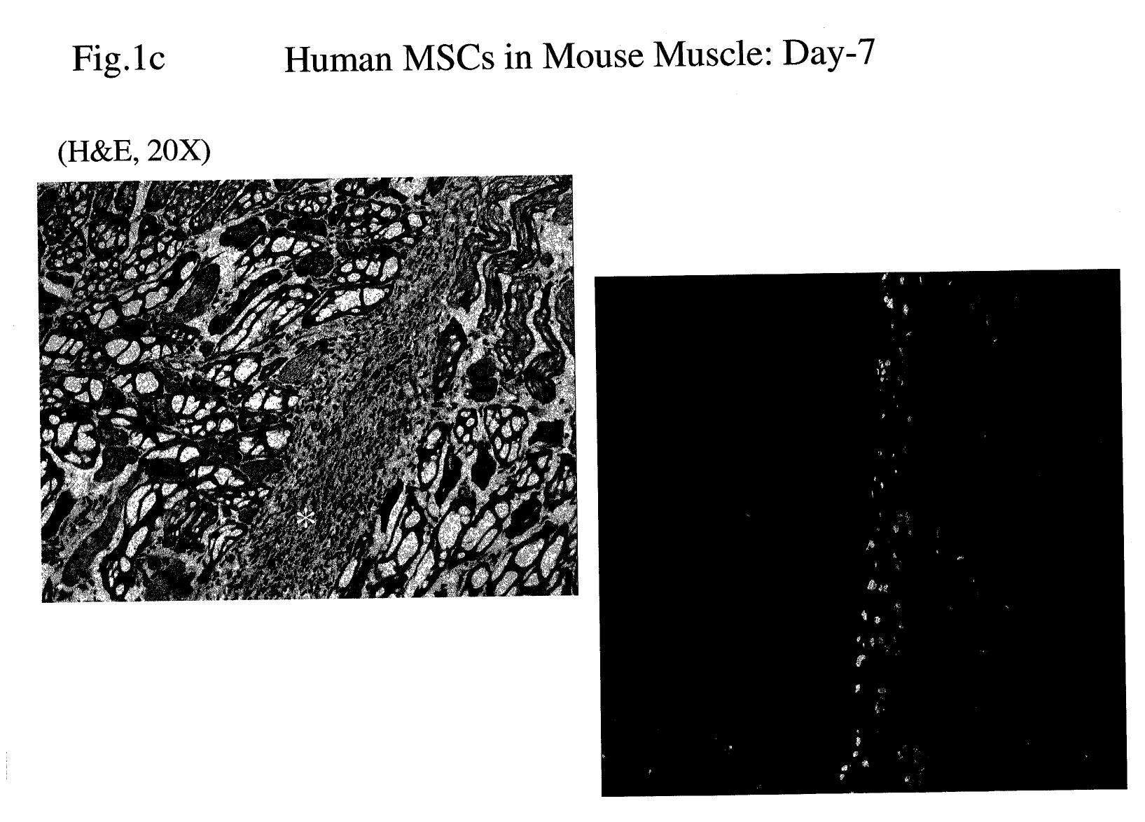

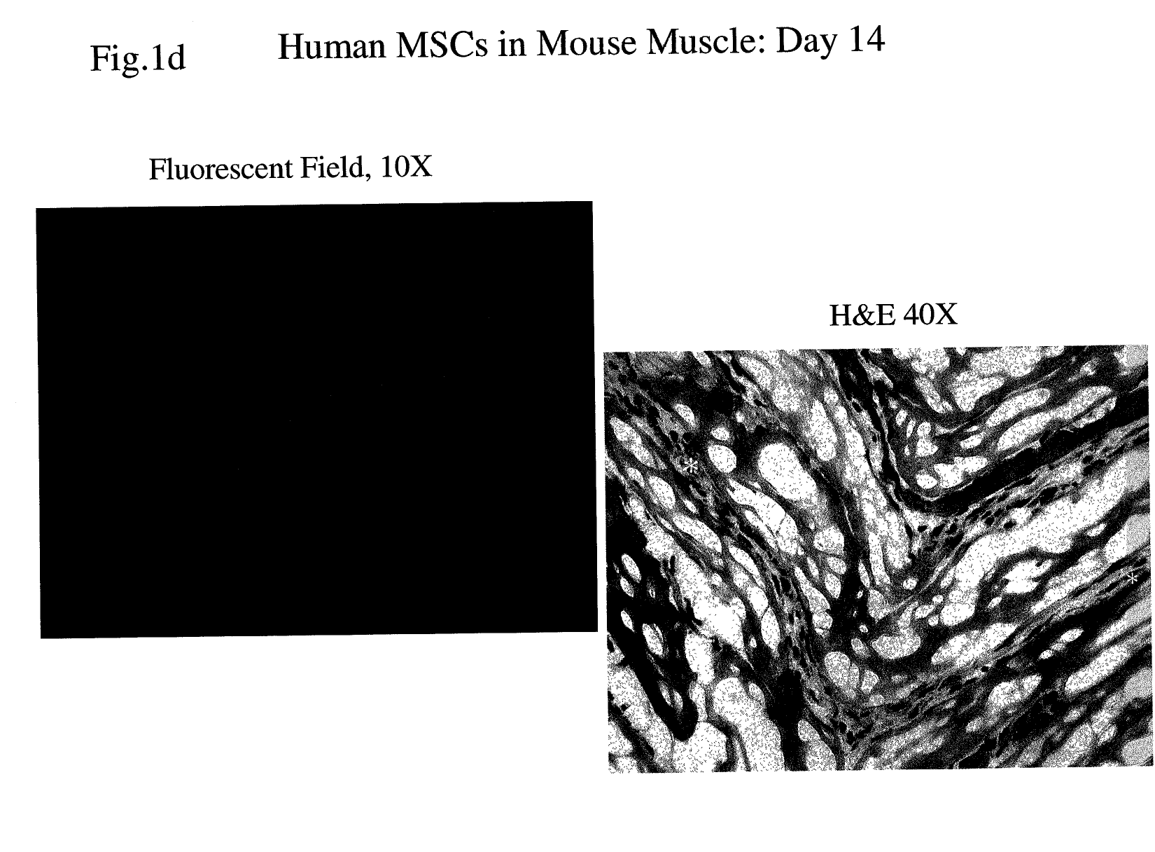

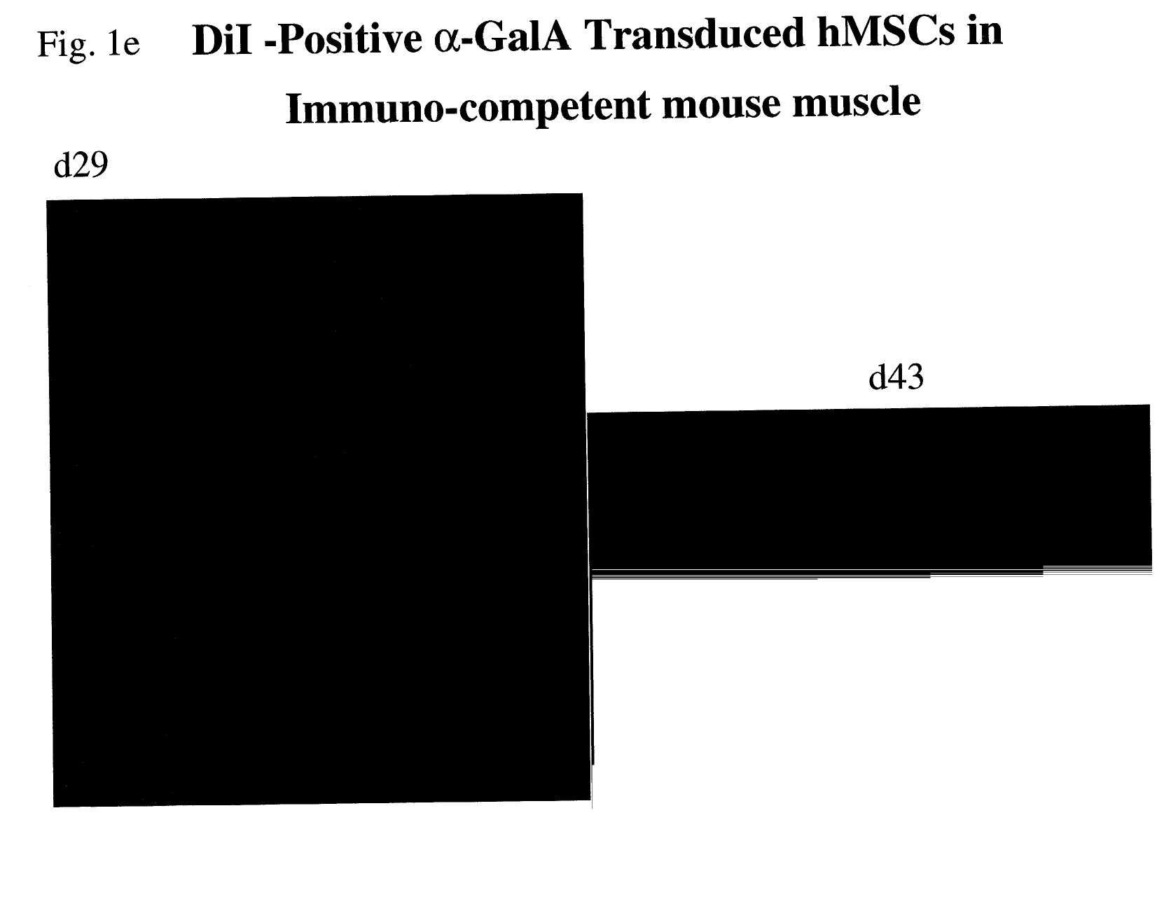

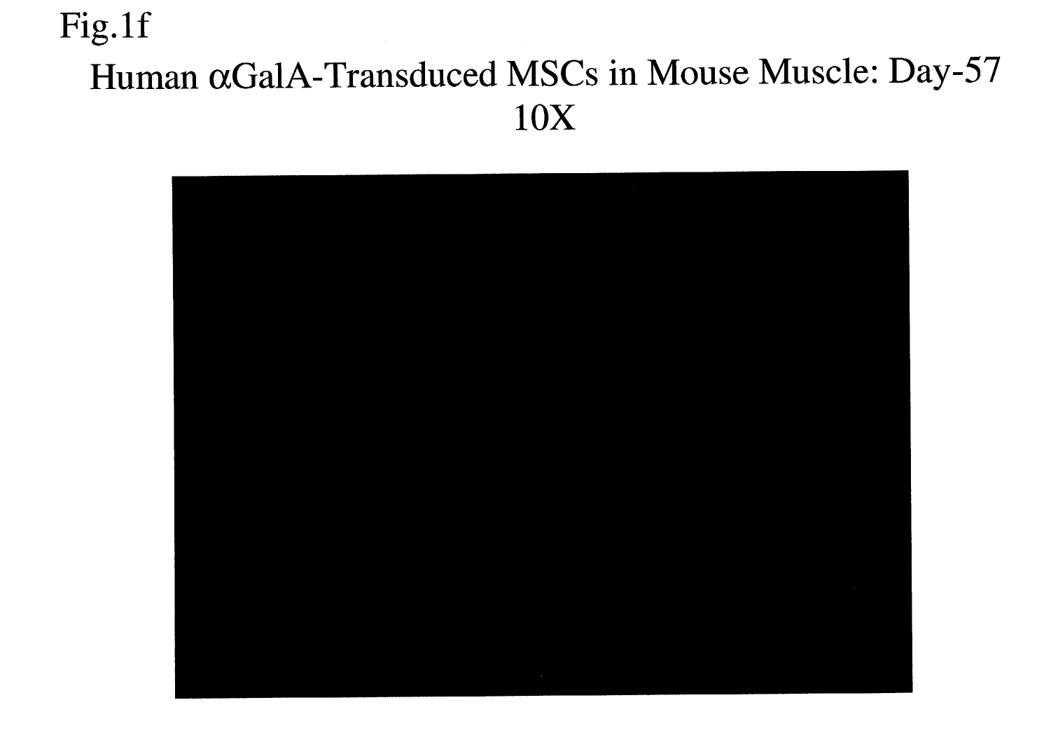

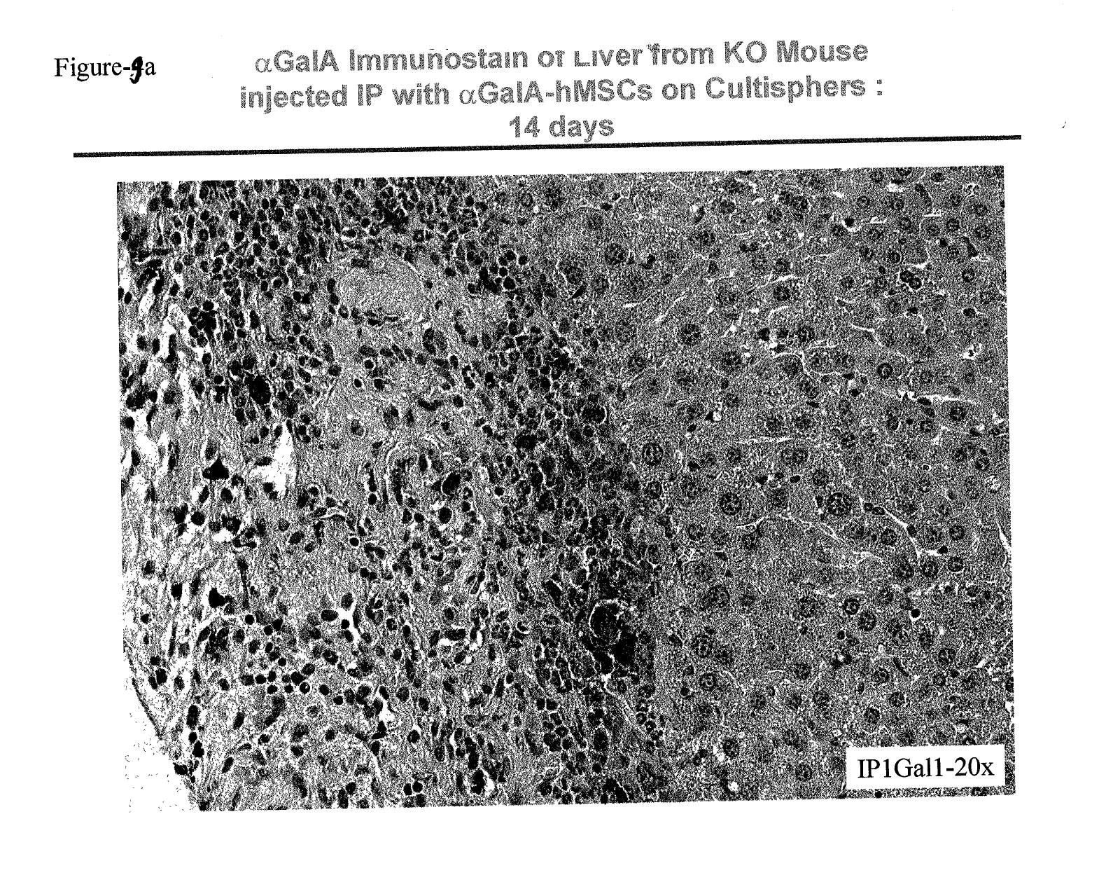

FIGS. 1A through 1F show representative images which show the presence of human mesenchymal stem cells in mouse muscle at 3 days (FIG. 1A), 7 days (FIGS. 1B and 10C), 14 days (FIG. 1D), 29 days and 43 days (FIG. 1E), and 57 days (FIG. 1F) subsequent to the administration thereof.

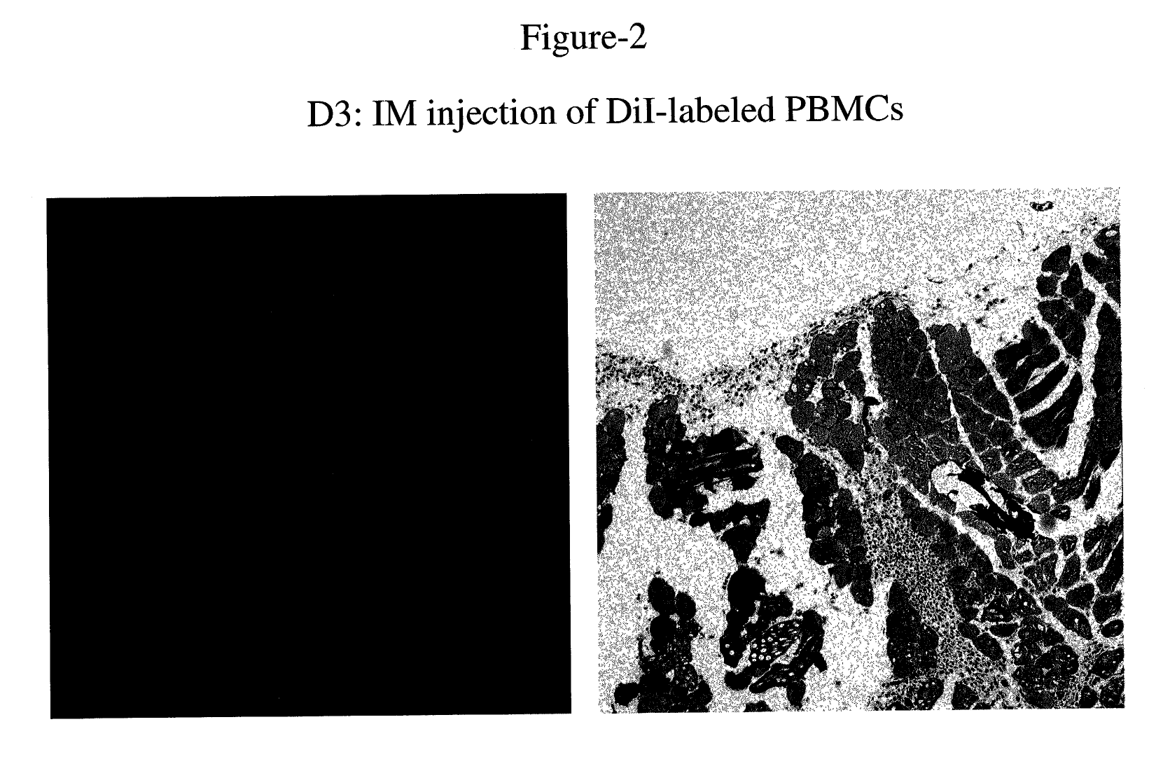

FIG. 2 shows images of sections of mouse muscle that were injected with human peripheral blood mononuclear cells, or PBMCs, three days subsequent to the administration thereof.

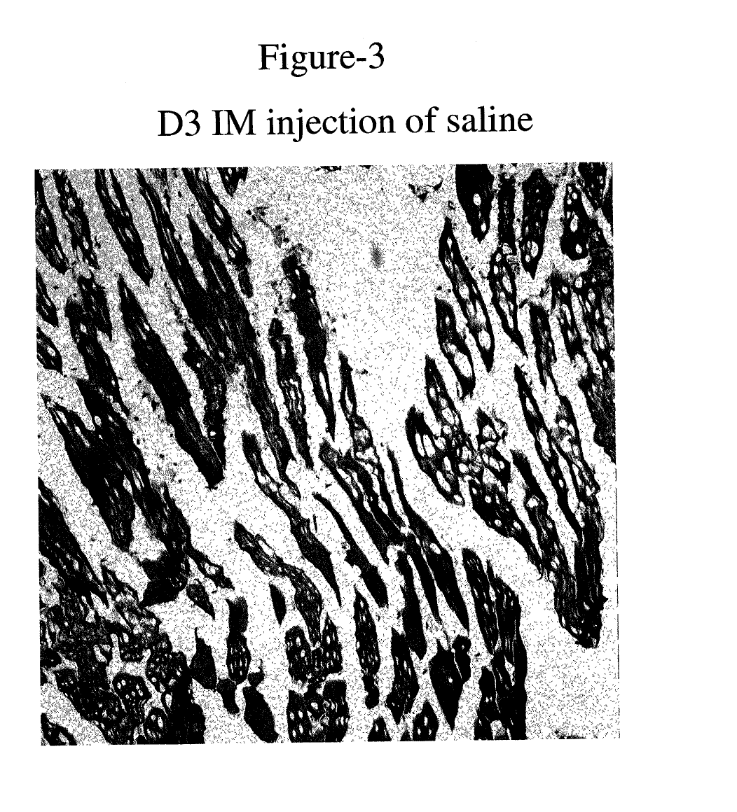

FIG. 3 shows an image of a section of mouse muscle that was injected with saline.

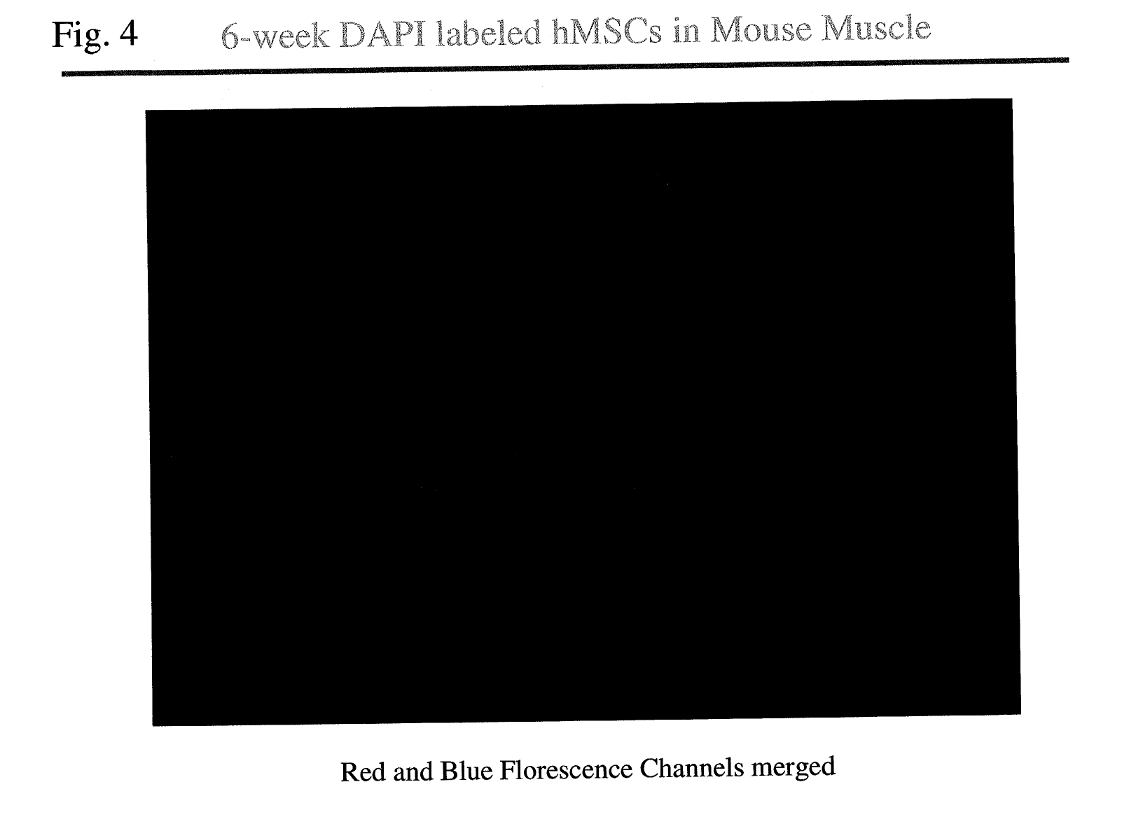

FIG. 4 shows DAPI labeled human mesenchymal stem cells in mouse muscle, 6 weeks after the administration thereof.

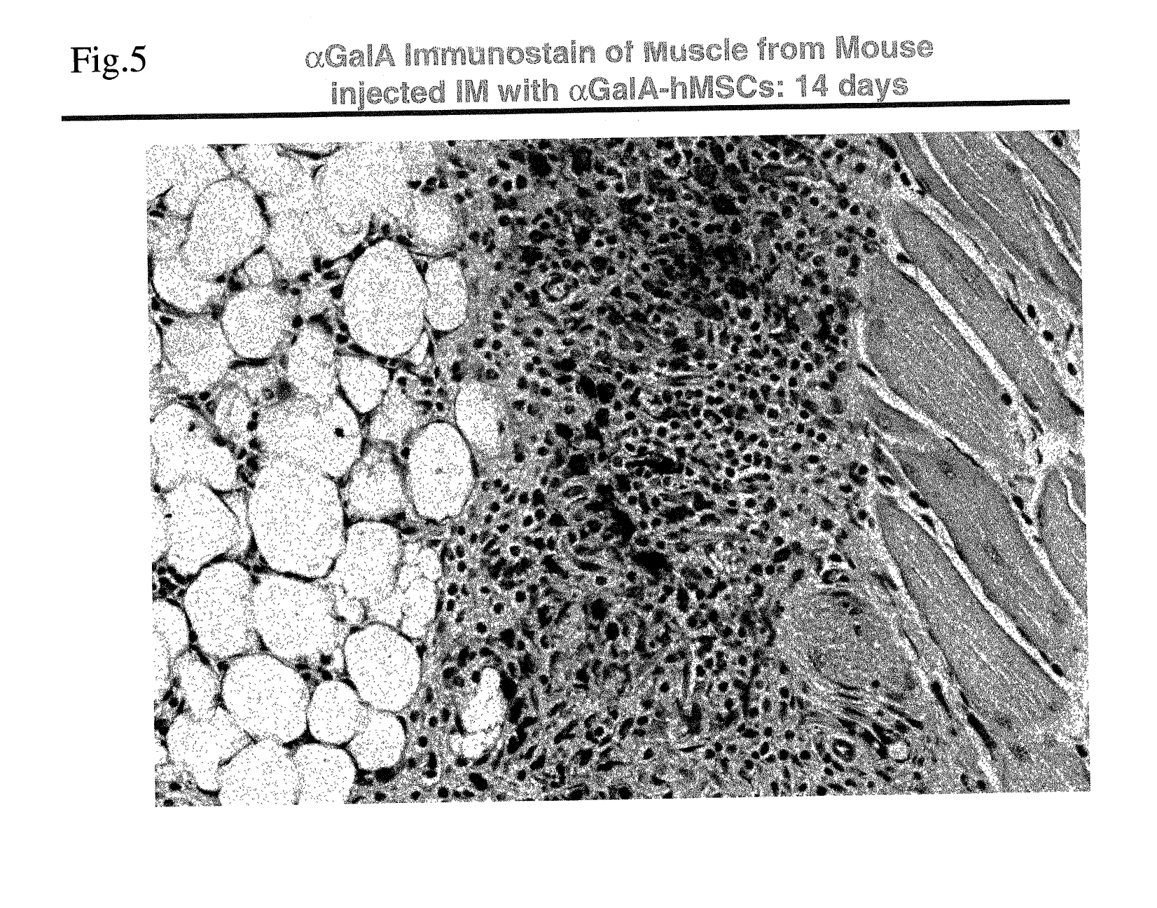

FIG. 5 shows an alpha-GalA immunostain of muscle from a mouse injected intramuscularly with human mesenchymal stem cells transduced with a vector including an alpha-GalA gene, 14 days after the administration thereof.

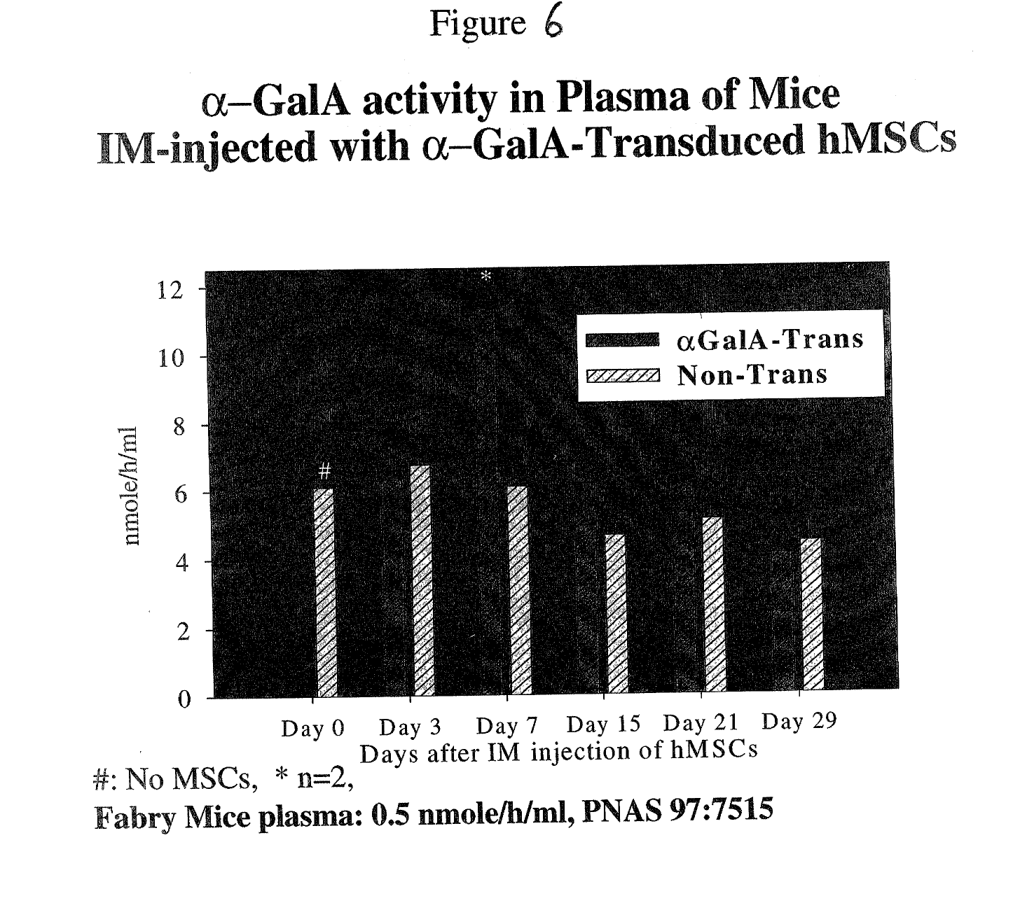

FIG. 6 is a graph showing alpha-galactosidase A (alpha-GalA) activity in the plasma of mice injected with human mesenchymal stem cells transduced with a vector including an alpha-GalA gene, as compared with mice that received human mesenchymal stem cells that did not include such vector.

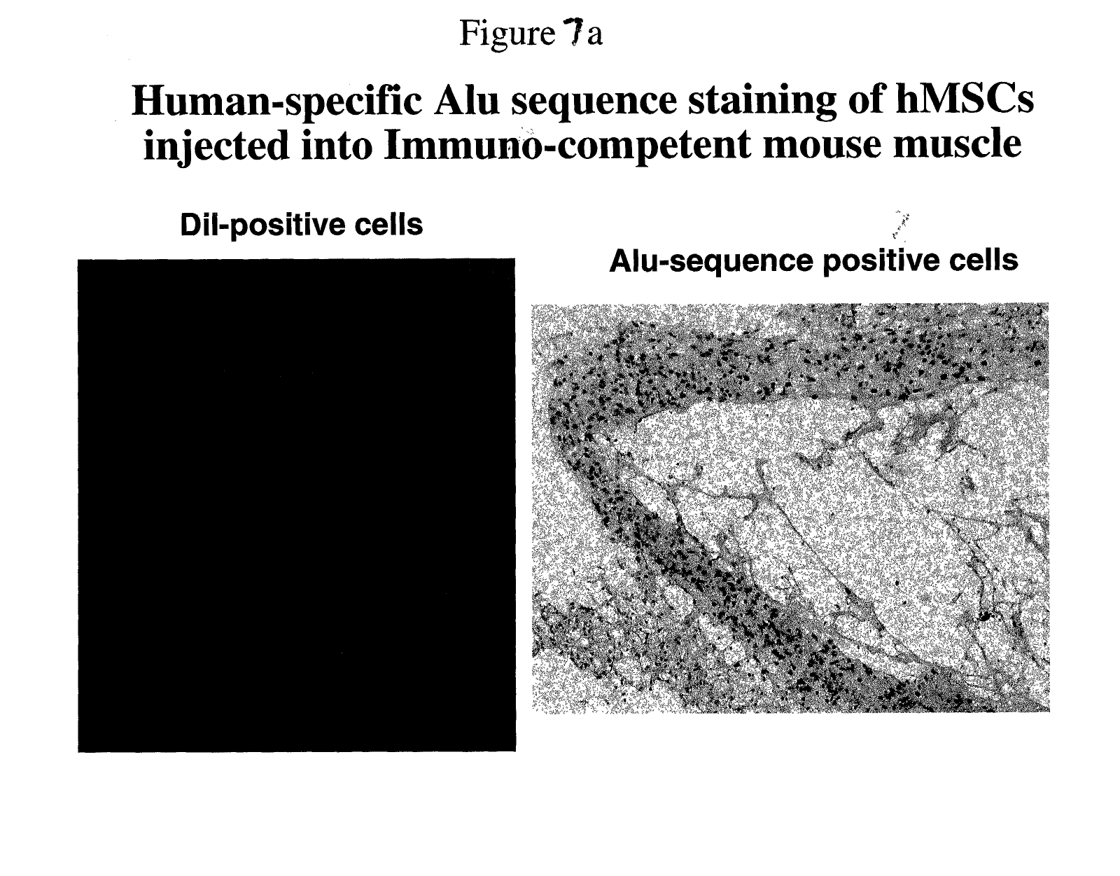

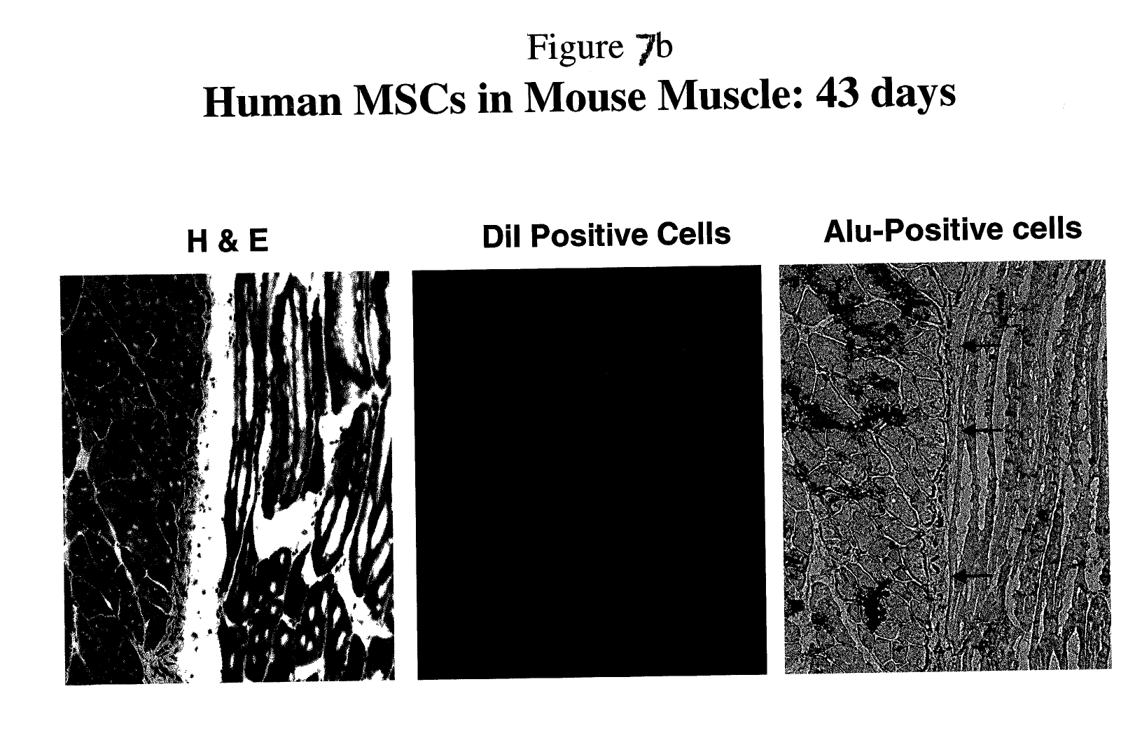

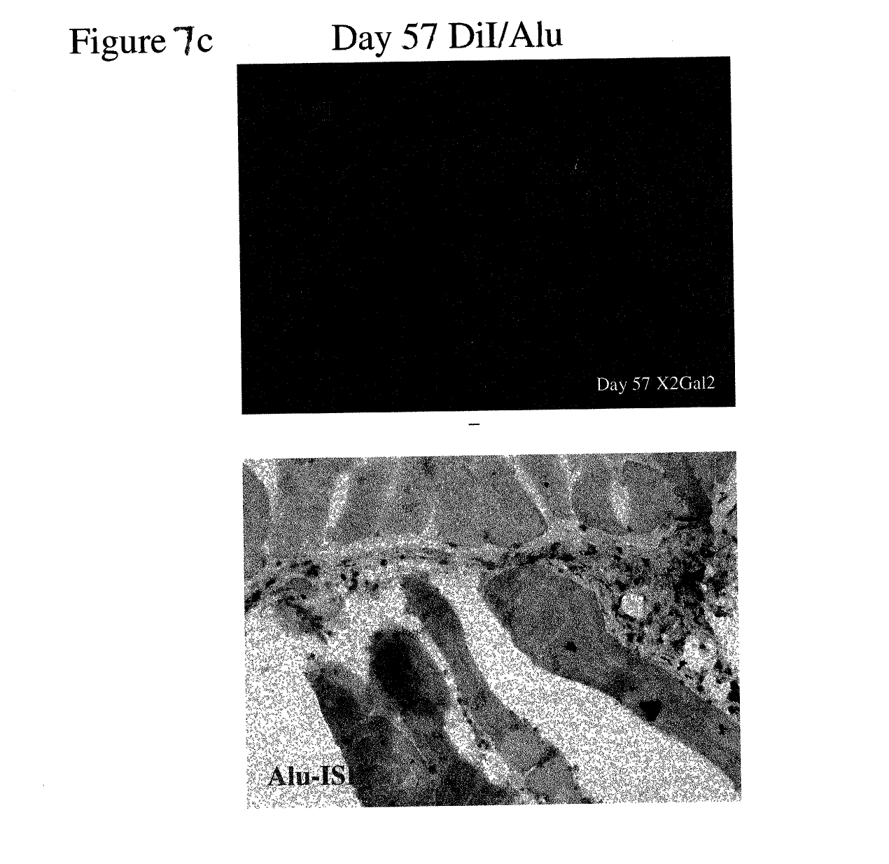

FIG. 7A shows human-specific Alu sequence staining of human mesenchymal stem cells injected into immunocompetent mouse muscle, three days after the administration of the human mesenchymal stem cells. FIG. 7B shows hematoxylin and eosin, Dil, and Alu staining of mouse muscle, 43 days after the administration of human mesenchymal stem cells to such mouse muscle. FIG. 7C shows Dil and Alu staining of mouse muscle 57 days after administration of human mesenchymal stem cells.

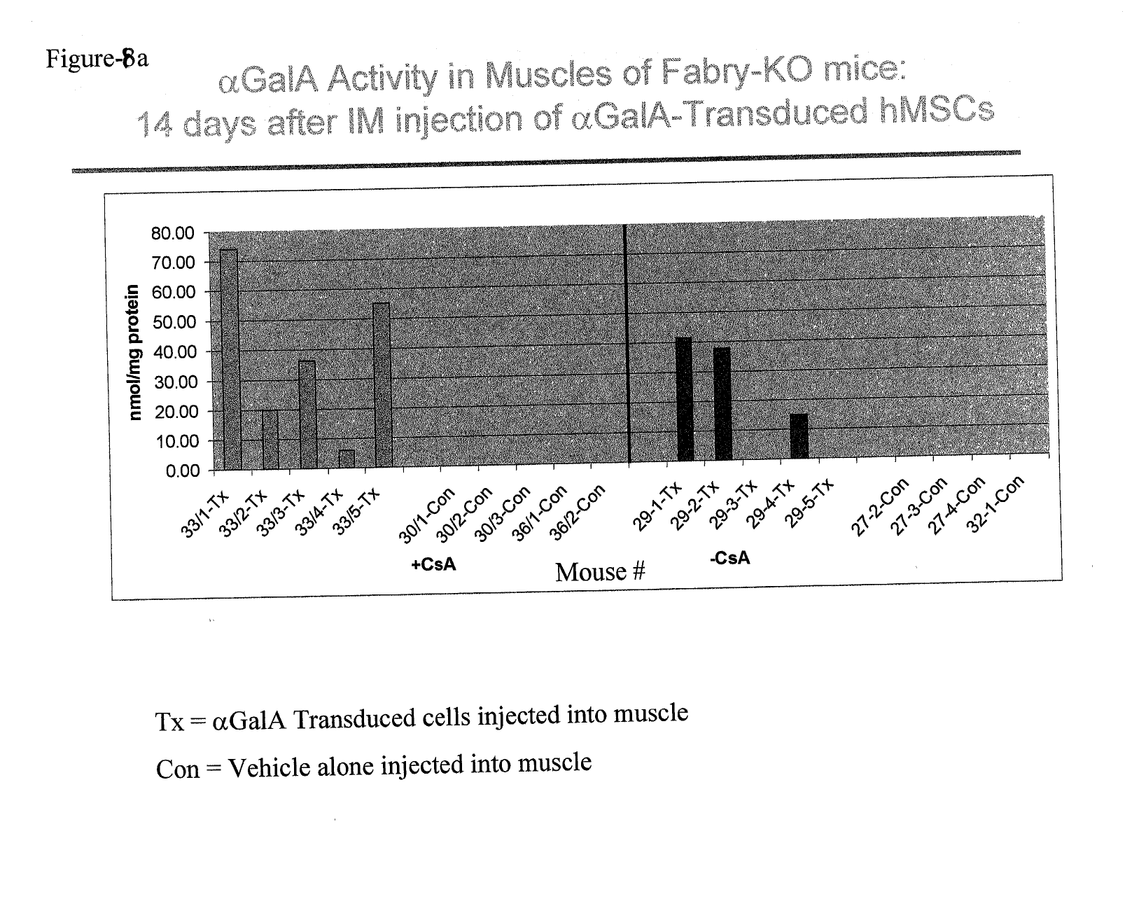

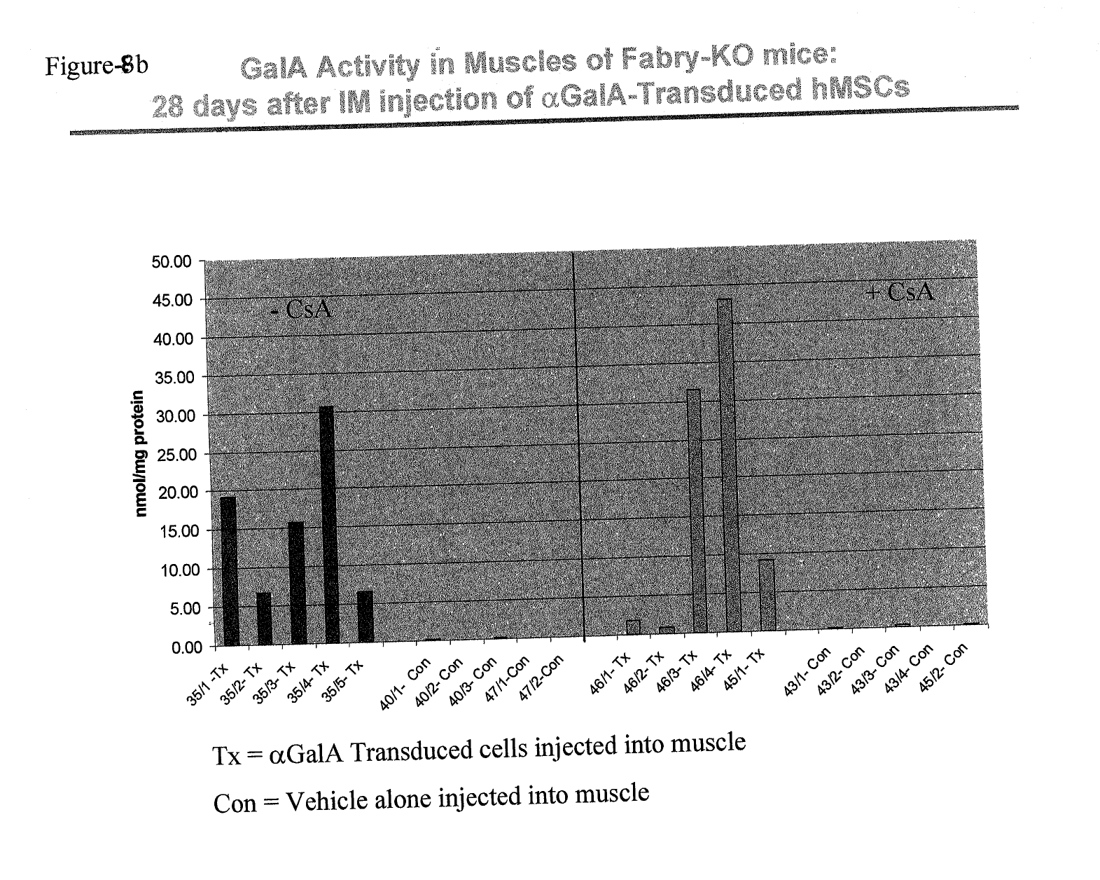

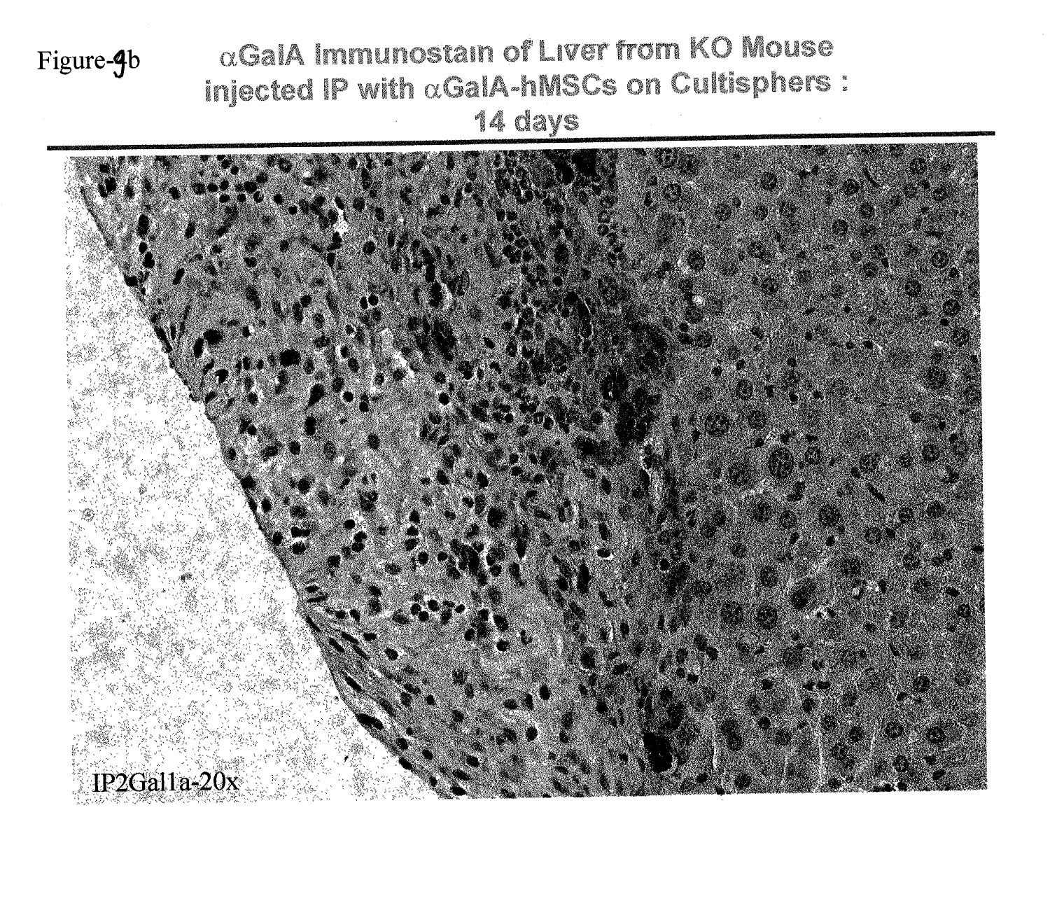

FIG. 8 shows alpha-GalA activity in Fabry knockout mice at 14 days (FIG. 8A) and 28 days (FIG. 8B) after intramuscular injection of human mesenchymal stem cells transduced with a vector including an alpha-GalA gene.

FIGS. 9A and 9B show alpha-GalA immunostains of liver tissue from a Fabry knockout mouse injected intraperitoneally with human mesenchymal stem cells transduced with a vector including an alpha-GalA gene.

FIG. 10 is a graph showing alpha-GalA activity in Fabry knockout mice that were injected intraperitoneally with human mesenchymal stem cells transduced with a vector including an alpha-GalA gene, as compared with control mice which did not receive human mesenchymal stem cells.

Applicants have discovered that xenogeneic mesenchymal stem cells can be administered, by injection or implantation, for example, into an immunocompetent post-natal animal. Thus, in accordance with an aspect of the present invention, there is provided an immunocompetent animal of a first species, which has had administered thereto, such as by implantation or injection, mesenchymal stem cells from an animal of a second species.

Animals of the first species include, but are not limited to, mice, dogs, pigs, rats, rabbits, baboons, and goats. In one embodiment, the animal of the first species is a mouse. In another embodiment, the animal of the first species is a dog.

Animals of the second species from which the mesenchymal stem cells are obtained, and which are implanted in the immunocompetent animal of the first species include, but are not limited to, rats and primates. In one embodiment, the primate is a human. In another embodiment, the primate is a baboon.

In a preferred embodiment, human mesenchymal stem cells are implanted into an immunocompetent mouse.

In another embodiment, rat mesenchymal stem cells are implanted into an immunocompetent mouse.

The mesenchymal stem cells may be implanted into the animal by a variety of methods known to those skilled in the art. Such methods include, but are not limited to, intramuscular injection, intravenous injection, intraperitoneal injection and intrahepatic injection. Alternatively, the mesenchymal stem cells, prior to implantation, may be seeded onto an appropriate support or matrix. The seeded support or matrix may be implanted surgically into an animal. Support or matrix materials which may be employed include, but are not limited to, poly (L-lactide), or PLLA and macroporouos gelatin microcarriers.

In another embodiment, the mesenchymal stem cells, prior to implantation, may have at least one exogenous polynucleotide encoding an agent of interest introduced therein while ex vivo. The genetically engineered mesenchymal stem cells then are administered to the animal, whereby the agent of interest is expressed in the animal. Agents of interest include, but are not limited to, alpha galactosidase A, soluble TNFRII-Ig and soluble IL-IRII-Ig. Once the agent of interest is expressed in the animal, it may be obtained from the animal by means known to those skilled in the art. The genetic engineering of mesenchymal stem cells is described more fully in U.S. Pat. No. 5,591,625, issued Jan. 7, 1997.

In yet another embodiment, the xeno-hybrid animal produced by the methods disclosed herein may be used in pharmaceutical research to establish drug effects, dosing parameters, specific toxicities, and multi-drug interactions. For example, a small molecule drug of interest may be administered to an immunocompetent xeno-hybrid post-natal animal in order to establish effects on human cells and tissues in an in vivo setting prior to the initiation of human clinical trials.

The invention now will be described with respect to the following examples; however, the scope of the present invention is not intended to be limited thereby.

### EXAMPLE 1

The objective of the present study was to investigate the behavior of human MSCs (hMSCs) in a xenogeneic mouse environment. The specific aims include 1) survival of hMSCs in an immunocompetent mouse, 2) survival of transduced hMSCs and expression of the transgene product by hMSCs in an immunocompetent mouse.

Materials:

hMSCs transduced with a gene encoding human alpha-galactosidaseA-FLAG (donor# 459)

hMSCs transduced with a gene encoding human Erythropoietin (donor# 219)

Mock or non-transduced hMSCs (donors 459 and 219)

CM-Dil (Molecular Probes Inc.)

C57BL/6 mice: 9 weeks old

Cyclosporine A (Sandimmune® Injection, Novartis)

Methods:

Transduction of hMSCs:

One group of hMSCs were transduced with alpha-Galactosidase A-FLAG (α-GalAFLAG). aGalA-FLAG was cloned in an MFG vector (pUMFG-aGalA-FLAG) obtained from J.Medin. (University of Illinois at Chicago) Ecotropic virus from AM12 cell line was used to transduce PT67 dualtropic packaging cell line. The viral supernatant was used to transduce hMSCs (donor # 459). Transduction was performed by centrifugation in the presence of protamine sulfate. Cells were transduced twice on consecutive days. Transduced cells were expanded in flasks and from P2 to P3 in a 10-stack factory. (Nunc) P3 cells were harvested and cryopreserved in liquid N₂.

A second group of hMSCs (donor #219) were transduced with EPO-GFP (Novartis, Propak virus) (Clinical Orthopedic Related Research, Vol. 379S, pgs. 571-590) expanded to P3 and cryopresrved.in liquid N₂.

Dil Labeling of Cells:

A stock solution of CM-Dil, 50 μg (Molecular Probes) was made by re-suspending in 100 μl of ethanol. On the day of injection into mice, transduced MSCs as well as non-transduced MSCs were thawed, washed once, and re-suspended at 10⁶ cells/ml in DPBS. The cell suspension was warmed to 37° C. CMDil was added to cells at a final concentration of 2 μM (100 μl CM-Dil stock solution to 23.7 ml of cell suspension). Cells were incubated at 37° C. for 2 min and then on ice for 2 min. Cells were centrifuged at 2000 rpm for 6-8 min and then washed with DPBS. The cell pellet was re-suspended in Optimem-1 medium at a concentration of 1 or 2×10⁶ cells per 50 μl.

Injection of Mice:

One group of mice received cyclosporine A daily, starting at day −1 for 14 days. Cyclosporine A was given at 25 mg/kg for 8 days and then at 20 mg/kg for 7 days. The stock suspension was diluted in sterile saline and injected intraperitoneally (about 250 pi per mouse).

On day-0, the Dil-labeled cells were delivered by intramuscular injection into the quadriceps femoris (thigh) muscle into groups of 3 mice as described in Table 1. The mice were anesthetized with nembutal (ip) prior to IM injection. 1×10⁶ Epo-transduced cells in 50 pi were injected into one site in the left thigh and 4×10⁶ agalA-FLAG cells in 100 μl were injected into 2 sites in the right thigh. For non-transduced cells, 1×10⁶ cells were injected into the left thigh and 4×10⁶ into the right thigh as in the case of transduced cells.

TABLE 1

| | | | Day of |
|---|---|---|---|
| Group | # of hMSCs | # of mice | Sacrifice |
| 1. | 4 × 10⁶ αGalA + 1 × 10⁶ EPO +/− | 3 + 3 | 3 |
| | cyclosporinA | | |
| 2. | 4 × 10⁶ αGalA + 1 × 10⁶ EPO +/− | 3 + 3 | 7 |
| | cyclosporinA | | |
| 3. | 4 × 10⁶ αGalA + 1 × 10⁶ EPO +/− | 3 + 3 | 14 |
| | cyclosporinA | | |
| 4. | 5 × 10⁶ control MSCs +/− cyclosporinA | 3 + 3 | 7 |
| 5. | 5 × 10⁶ control MSCs +/− cyclosporinA | 3 + 3 | 14 |

Schedule of Sacrifice:
Day 3: Sacrifice mice from group 1. Collect blood for αGalA, EPO activity and hematocrit.
Harvest muscle for histology.
Day 7: Sacrifice Groups 2 and 4. Collect blood for αGalA, EPO activity and hematocrit.
Harvest muscle for histology.
Day 14: Sacrifice Groups 3 and 5, collect both plasma and muscle for αGalA, EPO, hemotocrit and histology.

As described above the mice were sacrificed by CO₂ inhalation. Blood was collected by cardiac bleed with heparinized needle and syringe. Heparinized, Sure-seal capillary tubes were used to measure hematocrit in an AUTOCRIT Ultra3 machine. The cells were separated by centrifugation and plasma was stored at −80° C.

The left and right thigh muscles were dissected out, rinsed in DPBS and incubated in 20% sucrose for 20 min. The muscle was embedded in OCT compound and frozen blocks were prepared. Frozen muscle sections (7 microns) were observed under a fluorescence microscope or by confocal microscopy for the presence of Dil-positive cells. Some sections were counterstained with Sybr-green to stain nuclei. Adjacent sections were stained with hematoxylin and eosin.

The plasma was assayed for human Epo using an ELISA kit (R&D). Plasma was also assayed for alpha-galactosidase A enzyme activity with 4-methylumbelliferyl-α-D-galactopyranoside (Sigma) as substrate and N-acetylgalactosamine as an inhibitor of αGalactosidase B. One unit (U) of αgal-A activity hydrolyzed 1 nmol of substrate per hour at 37° C.

Results:

Histology:

Day 3: Many Dil positive cells were present at the injection site, in subcutaneous fat tissue and between muscle fibers. Fewer cells were seen in the left thigh compared to the right. H and E staining showed large MSC like cells between muscle fibers and in the subcutaneous tissue, some in clusters and others scattered. No sign of inflammation was noticed in any of the sections. No obvious differences were seen between cyclosporine treated and untreated mice.

Day 7: Same as in day 3, no inflammatory cells in all sections whether mice were treated with cyclosporin or not. Some sections had clusters of MSC-like cells by H&E that were also Dil positive cells. Again, cells were present in fat tissue, in the muscle and adjacent to tendon. Group 4 that included non-transduced cells also had many Dil-positive cells.

Day 14: Similar to early time points there was no inflammation. There appeared to be many more Dil positive cells in mice that received non-transduced cells than those that were injected with transduced cells; however, this was not a quantitative assessment of the number of cells. There was no obvious benefit from cyclosporine treatment.

Hematocrit and Systemic Human Epo Levels:

At day 3, hematocrits went up from 45.8% to 50% in mice that had no cyclosporine and 49.2% in mice that were treated with cyclosporine. At day 7 the hematocrits went up to about 65% with or without cyclosporine treatment. The levels were maintained at day 14.

Systemic Epo levels however showed a decline at day 7 from 1000 mIU/ml at day 3 to 30 mIU/ml in the absence of cyclosporine or 340 mIU/ml in the presence of cyclosporine treatment.

Alpha-Galactosidase A Enzyme Levels in Plasma:

αGalA enzyme activities were measured in the plasma. The results showed that the levels of enzyme activity were slightly higher at day 3 compared to day 7 in mice that received αGalA-MSCs. Mice that received non-transduced MSCs had levels similar to control mice that did not receive any cells. However, the levels of αGalA measured in the plasma are relatively low (at the lower limit of the assay) and in addition there is considerable endogenous activity in the mice.

Conclusions:

Human MSCs are not rejected by immunocompetent C57BL/6 mice. Dil-positive cells were seen up to 14 days in mice whether they received cyclosporine or not and whether the hMSCs were transduced or not. No signs of inflammatory cell invasion was seen at all the time points tested (day 3, 7 and 14). Expression of transgene product was seen systemically which declined with time.

### EXAMPLE 2

Materials & Methods

Isolation and Culture Expansion of MSCs:

Bone marrow samples were obtained from PureCell, L.L.C (CA). Human MSCs were isolated and cultured as previously described in Science, Apr. 2, 1999;284(5411):143-7. Human MSCs are also available from BioWhittaker, Inc. (Walkersville, Md.).

Transduction of hMSCs:

hMSCs were transduced with alpha-Galactosidase A-FLAG (αGalA-FLAG). αGalA-FLAG was cloned in MFG retroviral vector (pUMFG-αGalA-FLAG) described in (Human Gene Therapy, Vol. 10, pgs. 1931-1939 (1999)) and obtained from J.Medin (UIC). Virus was packaged in PT67 dualtropic packaging cell line (Clontech). The viral supernatant was used to transduce hMSCs. Transduction was performed by centrifugation as described in Clinical Orthopedic Related Research, Vol. 379S, pgs.571-590 and Mol. Ther., Vol. 3, pgs. 857-866 (2001) except that polybrene was replaced with protamine sulfate (15 μg/ml ). Cells were transduced twice on consecutive days. Transduced cells were expanded in flasks from P1 to P2 and from P2 to P3 in a 10-stack factory (Nunc). P3 cells were harvested and cryopreserved.

Dil Labeling of Cells:

A stock solution of CM-Dil (Molecular probes Inc.) was made by re-suspending 50 μg in 100 μl of ethanol. On the day of injection into mice, transduced MSCs as well as non-transduced MSCs or human PBMCs were thawed, washed once, and re-suspended in DPBS at 1×10⁶ cells/ml. Human PBMCs were isolated on a Ficoll-Hypaque gradient from leukopheresis samples. The cell suspension was warmed to 37° C. CMDil was added to cells at a final concentration of 2 μM (100 μl CM-Dil stock solution to 23.7 ml of cell suspension). Cells were incubated at 37° C. for 2 min and then on ice for 2 min. Cells were centrifuged at 2000 rpm for 6-8 min and then washed with DPBS. The cell pellet was re-suspended in serum free, phenol red free medium at a concentration of 1 or 2×10⁶ cells per 50 μl.

DAPI-Labeling of Cells:

hMSCs were suspended at 10⁶ cells per ml. A 1 mg/ml stock of DAPI (Tissue Culture Grade, Sigma) was made in sterile water. The cells were incubated in DPBS with 50 μg/ml of DAPI for 20 min at room temperature in the dark and washed extensively three times with DPBS. The labeled cells were then injected into the mice as described above.

Injection of Mice:

C57B16 mice were obtained from Jackson Laboratories and were 9 weeks old at the time of the experiment. One group of mice received cyclosporine A daily, starting at day −1 for 14 days. Cyclosporine A (Sandimmune® Injection, Novartis) was given at 25 mg/kg for 8 days and then at 20 mg/kg for 7 days. The stock suspension was diluted in sterile saline and injected intraperitoneally (about 250 μl per mouse).

On day-0, the Dil-labeled cells were delivered by intramuscular injection into the quadriceps femoris (thigh) muscle into groups of 3 mice as described below. The mice were anesthetized with nembutal (i.p.) prior to IM injection. 4×6 αgalA-FLAG cells in 100 μl were injected into 2 sites in the right thigh. For mice that received non-transduced cells, 4×10⁶ cells were injected into the into right thigh as in the case of transduced cells.

Some mice were injected with αGalA hMSCs at 2×10⁶ cells per leg. As a control for rejection of xenogeneic cells, human PBMCs were injected into muscles of a group of mice.

The mice were sacrificed by CO₂ inhalation at different time points. Blood was collected by cardiac bleed with heparinized needle and syringe. The cells were separated by centrifugation, and plasma was stored at −80° C.

Histology:

The thigh muscles were dissected out, rinsed in DPBS and incubated in 20% sucrose for 20 min. The muscle was embedded in OCT compound and frozen blocks were prepared. Frozen muscle sections (7 microns) were observed under a fluorescence microscope or by confocal microscopy for the presence of Dil-positive cells or DAPI positive cells. Some sections were counterstained with Sybr-green to stain nuclei. Adjacent sections were stained with hematoxylin and eosin (H&E).

For paraffin sections, muscles were fixed in 10% neutral buffered formalin and embedded in paraffin. Five micron sections were cut and processed for immunostaining with anti-αGalA.

Immunostaining for Human aGalA:

Polyclonal rabbit anti-human αGalA antibody was provided by Dr. Gary Murray, NIH. Antibody was used at a 1:500 dilution in Normal Goat Serum. The staining was either performed manually or on the Dako Autostainer Universal Staining System.

Slides were deparaffinized with xylene and then hydrated with a graded series of 100, 95 and 70% alcohol and finally in deionized water. Slides were incubated in Tris Buffer for 10 minutes, and endogenous peroxidase was blocked with 0.3% H₂O₂. Normal goat serum was used for blocking for 15 minutes. Diluted antibody was added and slides incubated for 30 minutes. Next the slides were incubated with biotinylated goat anti-rabbit antibody for 30 minutes followed by streptavidin-peroxidase for 30 minutes. Diaminobenzidine (DAB) was used as substrate to visualize the color. After counter staining with hematoxylin, the slides were dehydrated to xylene.

Measurement of αGalactosidase A Activity:

Plasma was also assayed for αGal-A enzyme activity with 4-methylumbelliferyl-α-D-galactopyranoside (Research Products International Corp.,Mt. Prospect, Ill.) as substrate and N-acetylgalactosamine as an inhibitor of αGalactosidase B (Sigma, St. Louis Mo.) as described in J. Biol. Chem., Vol. 253, pgs 184-190 (1978). One unit (U) of activity hydrolyzed 1 nmol of substrate per hour at 37° C.

In Situ Hybridization for Human-Specific Alu Sequences:

Frozen sections were prepared as above. The sections were observed for Dil positive cells and adjacent sections were stained with H and E. The sections were processed to detect human cells by in-situ hybridization with Fluorescein conjugated Human ALU probe (Innogenex, San Ramon, Calif.), and the ISH-Kit for Fluorescein labeled probes (Innogenex, San Ramon, Calif.) using the manufacturer's instructions. Additional Biotin and Avidin blocks (Innogenex) were used as recommended.

Detection of Anti-Human Xeno-Antibodies in Mouse Serum:

The sera from representative mice at different time points were tested for the presence of anti-human antibodies using FACS analysis. Briefly, test sera were diluted to 1:10 or 1:100 concentrations. Human MSCs were used as target cells. These cells were thawed, washed in serum containing medium, and then resuspended in 0.2% BSA solution at 0.4×10⁶ to 1×10⁶ cells/mi. 200 μl of cell suspension was added to 200 μl of test sera dilution. The cells were incubated for 1 h at room temperature. After washing with 0.2% BSA solution, the cells were incubated with 0.5 ml of 1:20 dilution of biotinylated horse anti mouse IgG+IgM for 1 h at room temperature. Biotinylated horse IgG was used for Isotype controls. The cells were washed and incubated with 100 μl of 10 μg/ml of Streptavidin-Alexa-Fluor 4888 conjugate for 30 min at room temperature. The cells were then washed in 0.2% BSA and resuspended in 0.2% BSA containing 0.5 μg/ml propidium iodide. Finally the cells were analyzed by flow cytometry. Viable cells (that did not take up PI) were gated and geometric mean of fluorescence in the FITC channel was tabulated.

Results

Histology:

Muscles from mice injected with hMSCs were prepared for frozen sections. Presence of Dil- or DAPI-positive cells was visualized by fluorescence microscopy in the red or blue channels respectively. Representative images showing the presence of hMSCs in the mouse muscle are shown in FIGS. 1a-1f. In muscles isolated 3 or 7 days after injection of MSCs, numerous Dil positive cells were present at the injection site (FIGS. 1a and 1c) in the subcutaneous fat tissue (FIG. 1a), adjacent to tendon (FIG. 1b), or in between muscle fibers (FIGS. 1a, 1b and 1c), H&E showed large MSC like cells between muscle fibers and in the subcutaneous tissue, some in clusters and others scattered. FIGS. 1b and 1c show confocal images of Dil-positive MSCs localized in the muscle that have been counterstained with the nuclear dye SYBR-Green. No significant sign of lymphocytic infiltration was noticed in any of the sections. Some sections showed the presence of neutrophils and or macrophages in localized areas. Other sections had eosinophilic cells in the area of the graft. An eosinophilic matrix was sometimes associated with the surrounding cells at the early time points (FIG. 1a). No obvious differences were seen between cyclosporine A treated and untreated mice. The picture was similar whether transduced or non-transduced cells were injected.

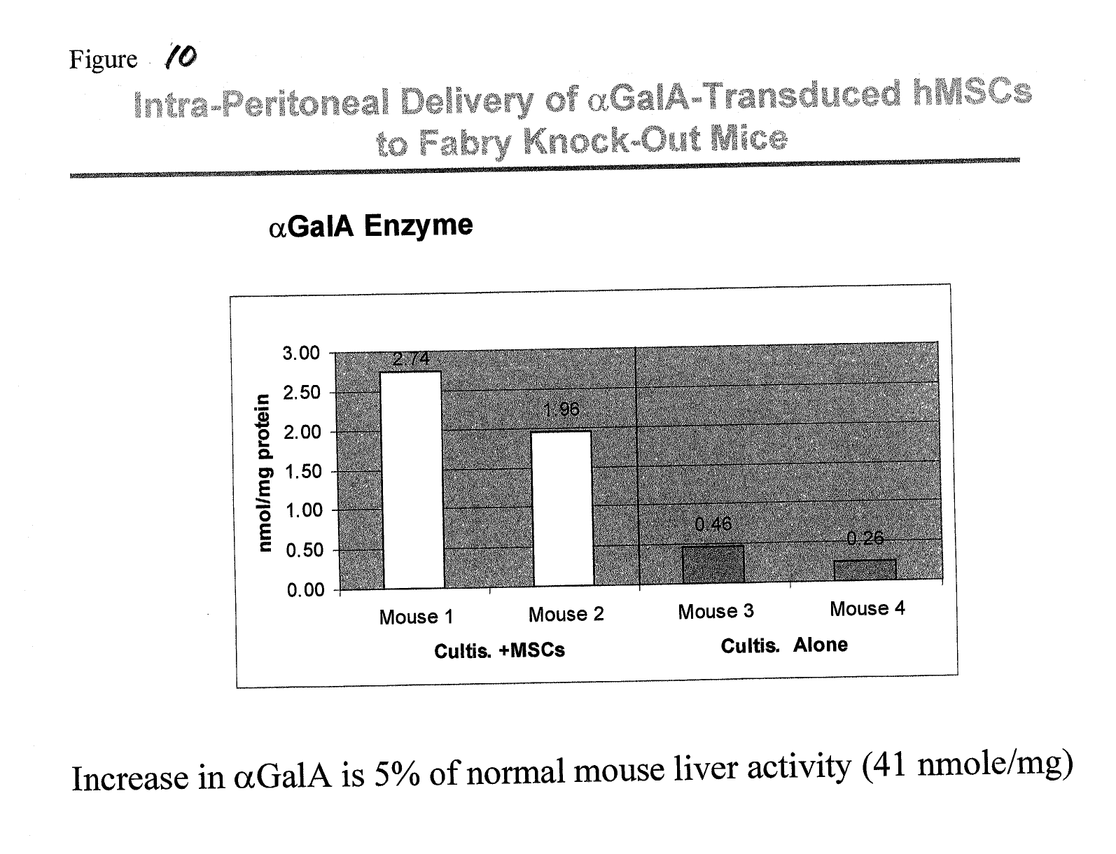

FIG. 1d shows numerous Dil-positive cells dispersed in rows between muscle fibers 14 days after injection. The adjacent H and E shows what appear to be MSCs in the corresponding areas with the absence of inflammatory cells. Again, no obvious benefit from cyclosporine A treatment was noticed. In a longer term study, numerous Dil positive cells were seen at day 29 (FIG. 1E). Sections from days 43 (FIG. 1E) and 57 (FIG. 1F) also contained many Dil positive cells again located adjacent to muscle fibers or in the interfascicular tissue.

Mice that received Dil-labeled PBMCs showed an area of lymphocytic cells at day 3 (seen by H and E) that colocalized with Dil-positive cells (FIG. 2). By day 15 negligible Di-l positive cells or inflammatory cells were seen. In case of muscles that received saline/vehicle, very few cells were seen in a small area of slightly damaged tissue (FIG. 3).

Because macrophages often have autofluorescence and could be mistaken for Dil-labeled cells, an alternative marking method was used that included DAPI labeling of the cells. DAPI intercalates in the nucleus and cells can be visualized in the UV channel. As seen in FIG. 4 numerous DAPI-positive cells are seen in the muscle, 6 weeks after injection. The figure shows a merged picture of blue fluorescence (DAPI) and Red fluorescence. Some of the red autofluorescing cells that could be macrophages appear as pink-purple in color.

Immunostaining of Muscles for αGalA:

Paraffin sections were made of muscles from mice injected with αGalA transduced cells. Immunostaining with antibody to αGalA showed the presence of numerous αGalA containing hMSCs. FIG. 5 shows the αGalA positive cells in the subcutaneous fat and also among the muscle fibers and in the interfascicular tissue adjacent to blood vessels and nerves.

Alpha-galactosidase A Activity in Plasma of Mice:

αGalA enzyme activities were measured in the plasma, FIG. 6 shows that C57BI/6 mice had baseline levels of αGalA activity around 6 nmole/h/ml. Mice that received αGalA-hMSCs showed a significant elevation in plasma αGalA at day 7 (2 out of 3 mice had levels of 24 nmoles/h/ml and 1 out of 3 mice had a value of 86 nmole/h/ml), however, by day 15 the levels were close to baseline. The elevation at day 7 was specific for mice that received αGalA-MSCs and was not seen in mice that received non-transduced MSCs.

In-Situ Histochemistry to Show Presence of Human Cells Positive for ALU-Sequence.

ALU-positive cells were present in large numbers at day 3. They were present at the injection site (FIG. 7a), scattered among muscle fibers close to and away from the injection site, and in the inter-fascicular tissue close to blood vessels, nerves and tendons. ALU positive cells were seen at all time points tested. Shown in FIG. 7b is a representative area at d 43 that shows a row of Dil cells that are also positive for Alu-sequence. FIG. -7c shows two fields of muscle from day 57 that contain Dil-positive cells that colocalize with Alu-positive cells.

Development of Xeno-Antibodies in Mice Injected with hMSCs.

Using flow cytometric analysis, it was shown that serum from mice injected with hMSCs contained antibodies to hMSCs. The antibodies were seen, however, at high concentrations of the serum indicating a relatively low titer. The antibodies decreased by six weeks.

Discussion

In this study it has been shown for the first time that hMSCs injected into the xenogenic environment of a post-natal immunocompetent mouse can survive and can express their transgene product.

hMSCs transduced with hEPO or αGalA were injected into the thigh muscle of C57BI/6 mice. We found that these cells survived in the muscle for as long as 8 weeks. This was assessed by Dil fluorescence. The presence of Dil fluorescent cells were present for such an extended period of time suggests that the hMSCs probably did not proliferate in the muscle. Although the percent of cells that persist was not quantitated, it appears that the number of Dil-labeled MSCs were reduced with time. It is not clear if the majority of cells die passively or if they move away from the site of injection. This apparent reduction of cells also occurs with syngeneic cells (results not shown) and may be an initial, innate host response to presence of exogenous cells in the muscle.

The muscles were stained with H & E for signs of inflammation. The results showed a variable response at the injection site. In many samples there was an obvious response to the injection, which included an eosinophilic matrix with cells. Most of the cells did not appear to be lymphocytic (small, mononuclear). Some of the cells looked fibroblastic, others appeared to be macrophages and in some cases eosinophilic cells were present. In certain muscles, pockets of neutrophils were observed which resolved by day 7 or latest by day 14. The results suggest that an influx of granulocytes and macrophages can occur as a response to the injury caused by injection or as a response to the abnormal presence of large numbers of cells that are not indigenous to the local environment. The absence of a lymphocytic infiltration suggests the lack of a specific anti-human cellular response. Studies with non-vascular grafts such as islets or skin, in mice have shown that the major mechanism of xenograft recognition is by indirect cellular responses, that is CD4 T-cells recognize xenoantigens processed and presented on self class 11 molecules by mouse antigen presenting cells (Murphy et al., Transplantation, Vol. 61, pgs. 1133-1137 (1996)). Rat islet grafts depleted of passenger leukocytes were rapidly rejected in mice, implying indirect xeno-recognition by T-cells (Wolf et al., Transplantation, Vol. 60, pgs. 1164-1170 (1995)). Neither CD4-dependent humoral responses that do develop after transplantation (Transplantation Proc., Vol. 25, pgs. 402-404 (1993)), nor CD8+ cytotoxic T cell responses were involved in the rejection of non-vascular xenografts (Transplantation Proc., Vol. 22, pg. 2335 (1990)). Based on these reports, it appears that although macrophages and neutrophils were present at the site of hMSCs in mouse muscle, they may not be involved in activating T cells for a xenogeneic rejection response as no lymphocytes were present and many hMSCs survived any innate macrophage-mediated assault that may have been mounted by the host.

The present study showed that the mice developed anti-hMSC xeno-antibodies (Table 2) albeit at a low titer.

TABLE 2
Anti-human xeno antibodies in mouse plasma

| Plasma sample | Xeno Ab | | |
|---|---|---|---|
| (1:100) | %+ | Mean FI | % PI positive |
| Control 1 | 8.7 | 34 | 6 |
| Control-2 | 8.8 | 36 | 5.4 |
| X1-NT-d7 | 95 | 69 | 6.3 |
| X1-NT-d14 | 94 | 73 | 9.6 |
| X2-NT-d15 | 10.3 | 34 | 7.1 |
| X2-NT-d29 | 90 | 66 | 6.1 |
| X2-NT-d43 | 60 | 47 | 8.6 |

1. Naive mouse plasma.
2. Xeno-Example 1: plasma from mice injected with non-transduced hMCSs (NT) collected at days 7 and 14 after injection.
3. Xeno-Example 2: plasma from mice injected with non-transduced hMSCs (NT) collected at days 15, 29, and 43 after injection.
FI: Flourescence Intensity
PI: propidium iodide (dead cells)

Although a specific cytotoxicity assay was not performed, the data suggest that the antibodies were not cytotoxic to hMSCs. As the percent of PI-positive cells was not increased compared to control sera. Others have reported that rejection of encapsulated xenogeneic cells was independent of antibodies and complement and mostly involved CD4 T cell mediated killing as described above.

The infiltration of neutrophils seen in some but not all experiments suggests contamination from injection through the skin or alternatively the presence of some dead cells in the injected population of hMSCs as a result of the manipulation. On the other hand this could be a reaction to the tissue damage from injection.

Significant numbers of αGalA positive transduced cells were detected at 2 weeks. The presence of hMSCs in mouse muscle was further confirmed by ISH with ALU probe specific for human DNA. Alu-positive cells were detected at 6 weeks and up to 8 weeks. In addition DAPI-labeled hMSCs were also present at 6 weeks.

The study also showed that not only do the hMSCs survive in mouse muscle, but transduced hMSCs also express and secrete the transgene product. Human erythropoietin was also expressed by hMSCs in injected into mice that caused a significant increase in the hematocrit that was sustained for 14 days, although plasma levels of Epo dropped significantly by 7 days in the absence of cyclosporine A treatment implicating the development of antibodies to the human Epo protein (results not shown). Also measured was αGalA enzyme in the plasma of mice that received αGalA-transduced hMSCs which reached a peak around d7. Although the serum levels of aGalA were modest and of short duration, elevated levels of aGalA enzyme activity for up to 4 weeks were found in the injected muscles of mice, whether or not the mice were treated with cyclosporine A (not shown).

### EXAMPLE 3

Continued Secretion of a GalA Enzyme from Explanted Matrix up to 4 Weeks (hMSCs Attached to PLLA) that was Implanted Subcutaneously

Poly (L-lactide), or PLLA, felt, purchased from Albany International Research Company (Mansfield, Mass.), was cut into 4 mm×5 mm×5 mm pieces, and the pieces were sterilized by hydrating in 100% ethanol for 30 minutes, followed by passing through an ethanol gradient (70%, 50%, 0% for 5 to 10 minutes each, followed by hMSC medium for 30 minutes). The pieces then were blotted on gauze and were placed in a 5 ml round bottom polypropylene tube.

Human mesenchymal stem cells (25×10⁶ cells/ml) were loaded onto the felt pieces and incubated for 2 hours on a roller table at 37° C. The pieces then were transferred to a 48 well plate, and then were implanted into mice on the back subcutaneously. One group of mice (3 for each time point) received 2×10⁶ cells per implant, and the other group of mice received 4×10⁶ cells per implant. The mice which received 2×10⁶ cells per implant were sacrificed at day 14 or day 28, and the implants were removed and evaluated for expression of αGalA. The mice were sacrified and the implants were excised from the back and placed in 1 ml of hMSC medium each and incubated for 24 hours at 370 and 5% CO₂. The medium was then collected, filtered through a 0.45 micron filter and frozen at −80° C. until measurement of αGalA enzyme activity as described in J. Biol. Chem., Vol. 253, pgs. 184-190 (1978). The mice which received 4×10⁶ cells per implant were sacrificed at day 12, and the amount of αGalA expressed from one of the implants was measured. The mean enzyme activity present in the medium is given in Table 3 below.

TABLE 3
Group 1-Loaded 2 × 10⁶ cells per implant (3 implants from 3 mice)

| | |
|---|---|
| Day 14 | 14.52 nmol/ml |
| Day 28 | 6.99 nmol/ml |

Group 2-Loaded 4 × 10⁶ cells per implant (1 implant)

| | |
|---|---|
| Day 12 | 53.61 nmol/ml |

### EXAMPLE 4

Persistence of aGalA Enzymatic Activity in Muscles of Fabry Knock-Out Mice Up to 4 weeks.

Fabry Knock-Out mice were injected intra-muscularly in the thigh muscle with aGalA-transduced hMSCs (Donor 475, transduced with VSV-G-pseudotyped retrovirus). 2×10⁶ cells were injected into each thigh. One group of mice received CsA, 25mg/Kg for 1 week and 20 mg/Kg for the second week intraperitoneally. The mice were sacrificed at 2 or 4 weeks. The muscles were dissected out and frozen. The tissue was processed in homogenization buffer (described in Proc. Nat. Acad. Sci., Vol. 97, pgs. 365-370 (2000), alpha-Galactosidase A enzyme measurement) using a Polytron and the lysate was then further sonicated. The tissue debris was centrifuged and the clarified lysate was used for aGalA enzyme determination. The αGalA enzyme activity was measured as described before and the activity was normalized to the total protein concentration determined with the BCA (Pierce Biochemical) kit.

FIG. 8 shows that the muscles injected with aGalA-hMSCs contained significant enzyme activity at 2 weeks (FIG. 8a) and 4 weeks (FIG. 8b). The control untreated muscles had close to null enzyme activity as expected from the Knock-Out mice. The sustained presence of enzyme activity in the tissues is evidence for the survival of hMSCs in the muscle. The half-life of recombinant enzyme in liver was previously reported to be less than 48 h (Proc. Nat. Acad. Sci., Vol. 97, pgs. 365-370 (2000).

### EXAMPLE 5

Presence of aGalA-Transduced hMSCs in Liver of Immunocompetent Mice After Intra-Peritoneal Injection of hMSCs Attached to Macroporous Gelatin Microcarriers (CultiSpher-G, Percell Biolytica, Sweden, Distributed by HyClone Laboratories Inc., Utah).

Approximately 5×10⁶ αGalA-transduced hMSCs (Donor 475, transduced with VSV-G-pseudotyped retrovirus) were attached to 25 mg of CultiSpher-G beads. The beads were hydrated and autoclaved as per the manufacturer's instructions. The cells and beads were incubated in hMSC medium for 2 hours. The medium was removed, cells were resuspended in phenol-free serum free DMEM-high glucose. The beads+cell suspension was injected intra-peritoneally into Fabry Knock-Out mice which have the aGalA gene functionally deleted (Drs. Roscoe Brady, Ashok Kulkami, NIH).

Two weeks later, the mice were sacrificed and the various organs were harvested. A piece of the central portion of the liver was fixed in 10% neutral buffered formalin. The tissue was embedded in paraffin and 5 micron sections were cut. The sections were stained with anti-aGalA antibody as described above for the muscles.

Results:

FIGS. 9a and b show numerous aGalA-positive cells in close proximity to the liver. Some of the positive cells appeared to be residing among the outer hepatocytes. The CultiSpher beads can be seen in the area undergoing degradation and probably also being resorbed by host phagocytic cells. Among other organs tested, the hMSC-CultiSphers appeared to have the most affinity for the liver. The presence of these cells was also detectable as αGalA activity in the liver lysate (FIG. 10).

The disclosures of all patents, publications (including published patent applications), depository accession numbers, and database accession numbers are incorporated herein by reference to the same extent as if each patent, publication, depository accession number, and database accession number were specifically and individually incorporated by reference.

It is to be understood, however, that the scope of the present invention is not to be limited to the specific embodiments described above. The invention may be practiced other than as particularly described and still be within the scope of the accompanying claims.

What is claimed is:

1. An immunocompetent post-natal animal of a first species having had administered thereto mesenchymal stem cells from an animal of a second species.

2. The animal of claim 1 wherein said animal of said first species is a mouse.

3. The animal of claim 1 wherein said animal of said first species is a dog.

4. The animal of claim 1 wherein said animal of said second species is a primate.

5. The animal of claim 4 wherein said primate is a human.

6. The animal of claim 4 wherein said primate is a baboon.

7. The animal of claim 1 wherein said mesenchymal stem cells include at least one exogenous polynucleotide encoding an agent of interest.

8. A method of making a xeno-hybrid animal, comprising administering to an immunocompetent post-natal animal of a first species mesenchymal stem cells from an animal of a second species.

9. The method of claim 8 wherein said mesenchymal stem cells include at least one exogenous polynucleotide encoding an agent of interest.

10. A method comprising isolating an agent of interest produced by the method of claim 9.

11. A method of testing a drug comprising administering said drug to the animal of claim 1.
